(19) United States
(12) Patent Application Publication
(10) Pub. No.: US 2024/0180725 A1
(43) Pub. Date: Jun. 6, 2024

(54) PROSTHESIS SOCKET SYSTEM AND METHOD FOR PRODUCING SAME

(71) Applicant: Ottobock SE & Co. KGaA, Duderstadt (DE)

(72) Inventors: Mario KOPPE, Duderstadt (DE); Lars Benjamin FINKE, Duderstadt (DE)

(21) Appl. No.: 18/553,082

(22) PCT Filed: Mar. 24, 2022
(86) PCT No.: PCT/EP2022/057812

§ 371 Date: Sep. 28, 2023

(30) Foreign Application Priority Data
Mar. 29, 2021 (DE) 10 2021 107 815.6

(51) Int. Cl. A61F 2/70; A61F 2/50; A61F 2/80

(52) U.S. Cl. CPC A61F 2/70; A61F 2/5044; A61F 2/80; A61F 2002/5007

(57) ABSTRACT

The invention relates to a prosthesis socket system having a main body (10) with a socket wall (11), which in the applied state surrounds a limb or a limb stump at least partially and has at least two opposite socket wall edges (12, 13) or socket wall regions (16, 17), wherein two anchors (22, 23) are provided, each anchor (22, 23) being arranged in a socket wall edge (12, 13) or on a socket wall region (16, 17), and a mechatronic functional element (20), which is fastened to the anchors (22, 23).

The invention relates to a prosthesis socket system having a main body with a socket wall, which in the applied state surrounds a limb or a limb stump at least partially and has at least two opposite socket wall edges or socket wall regions, and also to a method for producing such a prosthesis socket system.

A prosthesis socket is used to accommodate a limb or a limb stump in order to securely fasten further prosthesis components, such as joints, actuators, sensors, control devices and artificial limbs, for example prosthetic hands or prosthetic feet, to a patient. An essential point here is that a prosthesis socket is securely attached to the limb or limb stump without any risk of the prosthesis socket accidentally coming loose. Furthermore, the prosthesis socket ensures the orientation of the further prosthesis components relative to the limb or the limb stump. For this purpose, it is advantageous if the prosthesis socket lies as snugly as possible on the limb or the limb stump. To increase wearing comfort and to maintain a defined interface, use made, for example, of what is called liner technology, in which a prosthesis liner is arranged between the limb stump or the limb and the prosthesis socket. Locking between the liner and the socket can be effected by mechanical components or by vacuum. For so-called vacuum socket technology, it is necessary to have a closed prosthesis socket, which can be evacuated at least in part.

Prosthesis sockets are either individually shaped using a model or directly on the stump, or they consist of struts or wall components that are fastened to a distal endpiece in such a way as to be displaceable relative to each other. It is possible to move the struts or wall components relative to each other via straps or other devices.

DE 10 2007 025 410 A1 discloses a prosthesis socket for receiving an amputation stump and a limb with connecting means for a distal prosthesis device, wherein the prosthesis socket has at least one shell, which has a curved, open cross section and whose shell ends at least partially overlap each other in the applied state. At least one clamping means is arranged on the shell, which clamping means is effective in the circumferential direction and clamps the shell ends to each other.

EP 1 411 872 B1 discloses a prosthesis having a prosthesis liner with coupling pin and a prosthesis liner with longitudinal slits, on which a holder is arranged for connecting an artificial limb to the prosthesis socket. The prosthesis socket has a concentric collar in which a cylindrical adapter is mounted in a height-adjustable manner. The longitudinal slits are bridged, and the diameter of the prosthesis socket can be changed by means of clamping elements.

EP 1 555 967 B1 discloses a prosthesis socket having a sensor for measuring a pressure between the prosthesis socket and the amputation stump, and a sensor for measuring force, temperature and/or moisture. The sensors are connected to a display or a warning device. If limits for sensor values are exceeded, a warning signal is issued.

EP 3 454 792 B1 discloses a prosthesis socket having a proximal insertion opening and an inner circumference at least partially surrounding a stump, with at least one connection device for a prosthesis component, which can be fastened to the prosthesis socket, with at least one actuator, via which the inner circumference of the prosthesis socket can be changed, and with a controller which is coupled to at least one sensor, wherein the sensor is designed as an inertial sensor. The control device is connected to an actuator, wherein the actuator is activated or deactivated depending on the received sensor signals. On the prosthesis socket, an internal pressure sensor and/or motor current sensor for detecting the pressure applied by a support element to the stump can be arranged on a support element, wherein several support elements may be present, which are elastically formed or elastically supported. The support elements may be designed overlapping in the circumferential direction.

The object of the present invention is to provide a prosthesis socket system and a method for producing same, with which an improved overall care of the patient with mechatronic components can be provided.

According to the invention, this object is achieved by a prosthesis socket system having the features of the main claim and by a method having the features of the additional independent claim. Advantageous embodiments and developments of the invention are disclosed in the dependent claims, in the description and in the figures.

The prosthesis socket system having a main body with a socket wall, which in the applied state surrounds a limb or a limb stump at least partially and has at least two opposite socket wall edges or socket wall regions, is provided with two anchors, each anchor being arranged in a socket wall edge or on a socket wall region, and also a mechatronic functional element, which is fastened to the anchors and in particular is arranged between the socket wall edges or in a cutout within the socket wall. A defined mechanical interface between the mechatronic functional element and the socket wall or the socket wall regions of the prosthesis socket system is made available via the anchors in a socket wall edge or on a socket wall region. The individual design of the at least one socket wall is supplemented by a mechatronic functional element via the anchors, which element is designed as a prefabricated module. The mechatronic functional element can be prefabricated with all the components adapted to one another and fastened to the socket wall in order to complete the prosthesis socket. As a result, it is no longer necessary in particular for sensors and other components for providing an adaptive prosthesis socket or a mechatronic overall concept of the prosthetic care to be fastened individually in or on a socket wall and for the individual components to be individually configured. In the mechatronic functional element, all the electrical, electronic and mechanical interfaces and components can be industrially manufactured and integrated in advance, whereby the customization of the prosthesis socket system is only slightly restricted, if at all. The design of the remaining socket wall and of the other components of the prosthesis is still the responsibility of the orthopedic technician, to whom the system makes available a component with maximum, proven reliability and a minimum overall size.

In one development, the mechatronic functional element comprises at least one energy storage device, a sensor, a control unit, a communication interface and/or an actuator. By integrating the mechanical, electrical or electronic components and also the data processing equipment, all necessary assemblies and a central computing unit can be prefabricated and integrated into a module in a coordinated manner. The at least one energy storage device provides an integrated power supply including charge control for the mechatronic functional element. The electrical and electronic components can be arranged and accommodated in a watertight manner within the mechatronic functional element and can be provided with adequate mechanical protection by means of a housing or by appropriate integration. For example, the actuator allows the anchors to move toward or away from each other, thus deforming the socket wall or the main body. The main body or the socket wall can be formed elastically, such that it returns to an initial state after deformation or displacement from the latter. The socket wall or the main body can be flexible at least in part and can be brought into different shapes by the displacement of the anchors by the actuator or the actuators. This makes it possible, for example, to produce an improved adaptation of the socket wall to the limb or to a limb stump when applied.

Preferably, the mechatronic functional element is designed to be able to be reversibly fastened releasably to the anchors, such that the functional element can be arranged repeatedly between the socket wall edges or the socket wall regions and can be removed from there again, without components having to be destroyed. The reversible fastening can be effected, for example, by form-fitting and/or force-fitting elements, for example via hooks, pins, rails, clips, magnets, clamping devices and/or other mechanical or magnetic devices that allow the mechatronic functional element to be fastened and released.

The mechatronic functional element is designed for moving the anchors relative to each other, in particular for moving the anchors toward and away from each other, in order to achieve a change in the volume of the prosthesis socket. Another relative displacement of the anchors with respect to each other can also take place, such that a displacement of the socket wall, or of the socket walls with respect to each other, can take place by activation or deactivation of the mechatronic functional element. It is thereby possible to adapt the shape and in particular to adjust the volume of the prosthesis socket. For example, the volume can be increased in order to make it easier to put prosthesis socket on. When relevant loads or accelerations are detected, the volume can be reduced in order to ensure a secure fit of the prosthesis socket. Likewise in situations in which increased comfort is desired, for example when sitting. The mechatronic functional element can also be designed to be movable relative to the anchors, in order to permit a displacement of the mechatronic functional element on the socket wall or relative to the socket wall. Even without changing the position of the anchors with respect to each other, the position of the socket wall can be changed by activation of the mechatronic functional element, for example in order to position sensors at a different location, to cause a shift in pressure or to obtain a modified stiffness through the new positioning of the mechatronic functional element.

The anchors are advantageously designed as form-fitting elements and/or force-fitting elements in order to permit reversible fastening of the mechatronic functional element to the socket wall edges or to the socket wall. The anchors can be designed, for example, as rails, rail segments, pins or holes, optionally also as bores in the socket wall or in the socket wall region. A positive locking mechanism can be obtained via the rails, by inserting a corresponding rail or guide on the mechatronic functional element. The rails can be designed as rail segments and can be arranged or formed only in some regions along the socket wall edges. Likewise, form-fitting elements or projections or undercuts can be clipped into the rails so that there is no pushing insertion movement in the longitudinal extent of the rail, but instead a relative movement perpendicular thereto. Clamping, i.e. positive coupling, can also take place in rails or clamping elements. Additional holding forces can be provided via magnets and, for example, form-fit securing elements can be brought from an unlocking position to a locking position when a desired end position is reached.

The anchors can be fastened in or on the main body in a form-fitting, force-fitting and/or cohesively bonded manner. Alternatively, the anchors can be designed as part of the socket wall, of the socket wall edge or of the socket wall edge region. The anchors can in particular be cast, clamped, hooked, glued, laminated or pushed into the socket wall. Similarly, the anchor or the anchors can be screwed, welded, riveted, glued, laminated, sewn or pushed onto the main body. The anchors can also be designed as bores or molded projections or slots and also as undercuts.

In one variant, the main body is individually shaped, for example modeled directly on the limb or on the limb stump. Alternatively, the main body can be formed from prefabricated modules, either standard modules or individually shaped modules, or can be formed as a standard main body with different assembly sizes. The materials used are selected according to the manufacturing process and the desired properties of the prosthesis socket.

The socket wall edges can be created by a separating process, for example by being cut out, lasered out, punched out separated in some other way. If, for example, free regions are already present in the main body, the socket wall edges can be adjusted by grinding or milling such that the mechatronic functional element can be positioned between the socket wall edges or fastened between the anchors in such a way that the mechatronic functional element is fastened on the mutually opposite socket wall edges. The mechatronic functional element can cover the region between the mutually opposite socket wall edges or can completely or at least partially fill this. The mechatronic functional element can be arranged in a free space or a cutout between the socket wall edges in such a way that a closed or substantially closed cross section and thus a completely circumferentially closed prosthesis socket can be achieved.

The socket wall edges are spaced apart from each other and at least in part oriented along each other, i.e. substantially parallel to each other or with slight misalignments to each other. Alternatively, the socket wall edges can widen conically or curved in the longitudinal extent of the main body, such that a functional element, in particular by insertion in the proximal-distal direction, can be easily positioned in relation to the respective anchors and fastened to the main body.

In one embodiment, the socket wall edges extend in the proximal-distal direction of the prosthesis socket or are oriented obliquely to the proximal-distal direction, as a result of which the assembly from the direction of the proximal socket edge is facilitated.

The main body can be formed as an injection-molded component from thermoplastic material, additively manufactured or manufactured in a laminate structure. A substantially conical basic shape of the main body is preferred, in particular provided with an open cross section, wherein the open cross section or the recess for introducing the mechatronic functional element can be produced either during the manufacture of the main body or at a subsequent stage. Within a conical basic shape, whether it is with an open cross section or with a closed cross section, the main body can have at least one cutout to accommodate the mechatronic functional element therein or so that the mechatronic functional element can at least partially fill and/or cover the cutout.

An end cap can be formed on or fastened to the main body. The end cap can be of a closed or partially closed design and, at the distal end, can have fastening devices for further prosthesis components, in particular for joint devices, bridging components such as lower-leg tubes or forearm tubes, or prosthetic lower legs or prosthetic forearms.

In one embodiment, fastening devices for the mechatronic functional element are arranged on the end cap, via which devices it is possible to lock the mechatronic functional element to the end cap. The fastening devices can act in a form-fitting and/or force-fitting manner and in particular support the correct positioning of the mechatronic functional element in or on the main body.

The socket wall edges can be designed to be elastically displaceable with respect to each other, and in particular they can be displaced toward each other and away from each other in order to permit a volume adjustment via an actuator in the mechatronic functional element. On account of the elastic displaceability, the return to a starting position is facilitated by the elastic restoring forces. Alternatively or in addition, the socket wall is designed to be reversibly deformable, so that different shapes or volumes can be adjusted by a corresponding displacement of the anchors on account of the actuators.

In one development, fastening devices for the anchors are arranged or formed on the mechatronic functional element, such that the mechatronic functional element is able to be fixed to or in the anchors. The fastening devices are, in particular, correspondingly configured form-fitting devices in which the anchors engage or with which the anchors come into engagement when the mechatronic functional element is mounted.

In one embodiment, the mechatronic functional element is arranged between the socket wall edges, in a cutout of the socket wall or on the outside of the socket wall and supplements the main body for the configuration of a complete prosthesis socket.

The method for producing a prosthesis socket system, as described above, initially provides for the creation of a main body with a socket wall and the creation of socket wall edges. The socket wall edges can be created during the production of the main body or at a subsequent stage. Furthermore, anchors are also to be formed on or fastened to the socket wall edges or socket wall edge regions. The one-piece design of anchors takes place during the production of the main body, and if separate anchors are used, these are fastened to the main body, in particular to the socket wall edges or socket wall edge regions. The anchors can lie opposite each other and can be designed, for example, as rails or guides. A mechatronic functional element is then fastened to the anchors, and thus a prosthesis socket is supplemented to give a mechatronic total system. In one development, if a distal end cap is arranged or formed on the main body, the mechatronic functional element is fastened to the end cap, in particular to prevent displacement in the proximal direction.

The main body can be produced by means of an additive manufacturing process, for example after detection of the stump contour and adaptation of the necessary data to the loads and to the intended use. Alternatively, in the context of an injection molding process, the main body can be molded from a thermoplastic material in a different way, or it can be produced by a lamination process. In principle, it is also possible for it to be formed directly on a model, for example a cast, or on the limb stump. Here too, the socket wall edges or the socket wall edge regions can be formed during the production of the main body or at a subsequent stage.

The configuring of a main body, for example around a plaster model, is facilitated in the context of a lamination process, or an application of a thermoplastic material, if the main body is formed with a closed cross section. The plaster model or the other model is completely surrounded circumferentially by the socket wall material. The socket wall edges are then formed by separating out a segment of the main body.

In one development, the anchors and the base body are connected to each other in a form-fitting, force-fitting and/or cohesively bonded manner, for example cast in, screwed on, welded on, riveted on, clamped in, hooked in, glued in, laminated in, laminated on, sewn on, pushed in and/or pushed on and, if necessary, thereafter cohesively bonded, for example by welding, gluing or the like.

In one development, the mechatronic functional element is locked onto the anchors by form-fit and/or force-fit engagement, in order thereby to permit a reversible fastening and a reversible release of the respective functional element.

By designing the mechatronic functional element as a prefabricated module with all the necessary assemblies and with a central computing unit, it is possible to achieve an optimal coordination of all the components, such as the energy supply, sensors, actuators, and open-loop and closed-loop controls. With the prosthesis socket system, it is possible to create an active, situation-related volume adjustment of an individual prosthesis socket with a standardized functional group. In addition, there are standardized interfaces for networking with several mechatronic modules. For example, several mechatronic functional elements can be used on a main body between two socket wall edges, either to increase the adjustment distance or to allow different adjustments. Likewise, communication between different prosthesis socket systems or to an evaluation device or an orthopedic technician can be established in order to transmit usage data and to enable an evaluation.

The prosthesis socket system provides a combination of a prefabricated mechatronic functional element and, if appropriate, individual components of a prosthesis socket that are adapted to the respective limb and the respective patient. On account of the predefined external dimensions of the mechatronic functional element, it is possible to ensure precise manufacture of the prosthesis socket and at the same time to achieve individual adaptability. Thus, it is no longer necessary to produce a rigid, circumferentially closed individual socket and to compensate for volume fluctuations, due to a reduced service life or a change in the physical condition of the prosthesis user, by adjustment of a liner system. The manufacture of the prosthesis socket can be facilitated, since the manufacturing precision no longer has to be so high. All critical mechatronic interfaces can be manufactured industrially in advance, resulting in maximum reliability during use, along with a minimum overall size.

Exemplary embodiments and aspects of the invention are explained in more detail below using the accompanying figures. In the figures:

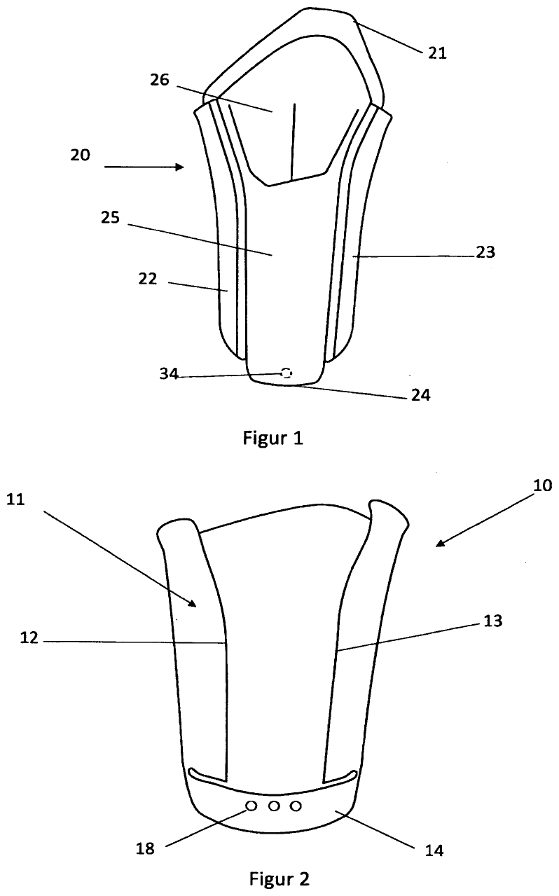

FIG. 1 shows a single view of a mechatronic functional element;

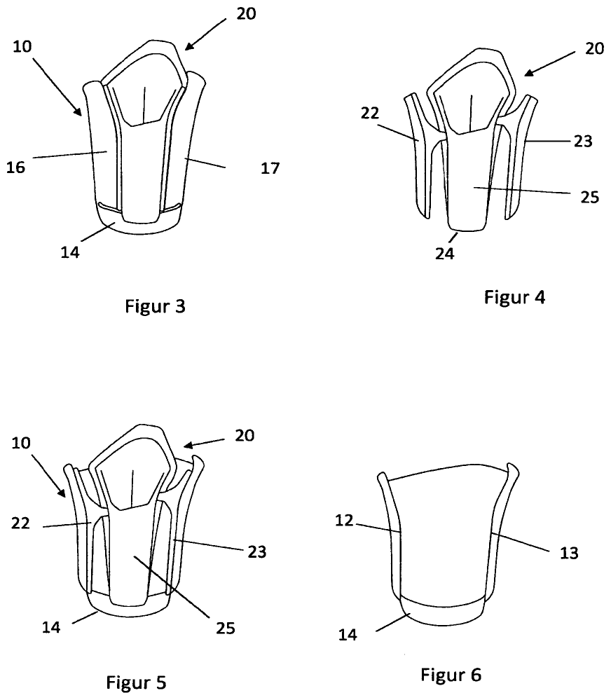

FIG. 2 shows a single view of a main body;

FIG. 3 shows a prosthesis socket system with a main body and an inserted functional element;

FIG. 4 shows a functional element with displaced anchors;

FIG. 5 shows a variant of FIG. 3 in an open state;

FIG. 6 shows a main body with an enlarged diameter;

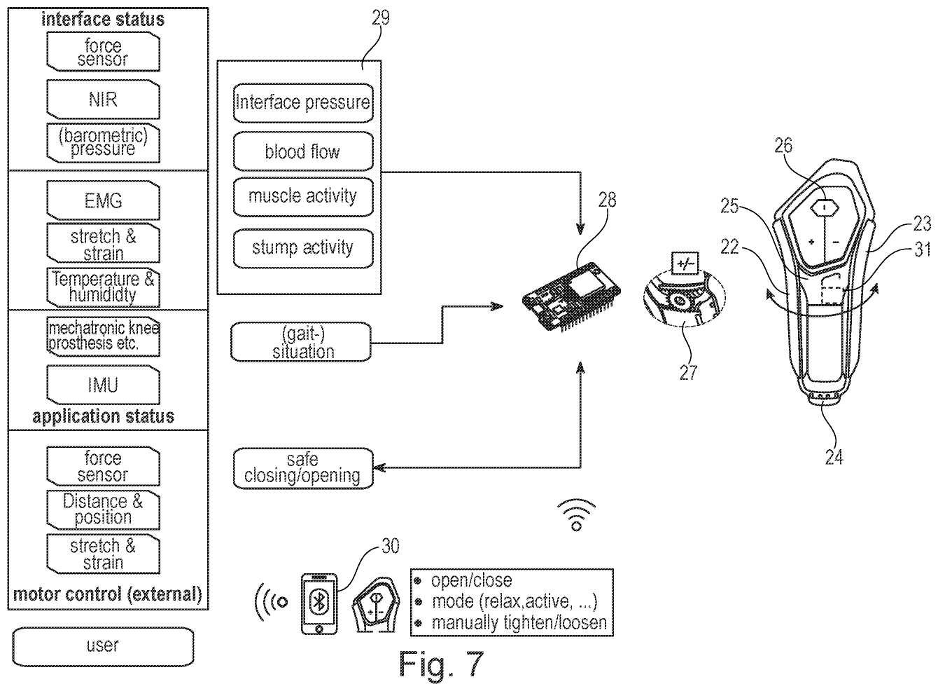

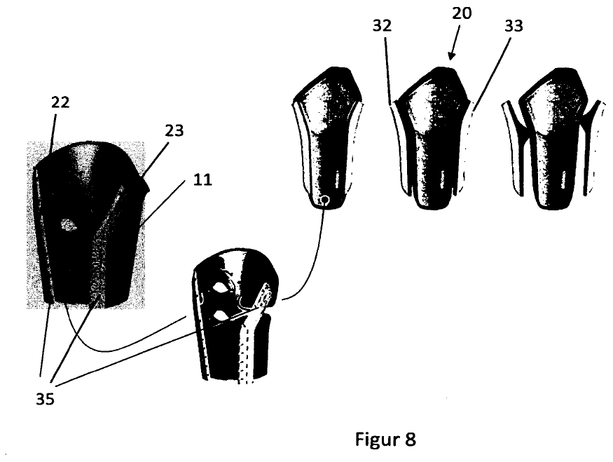

FIG. 7 shows the functional element with additional components;

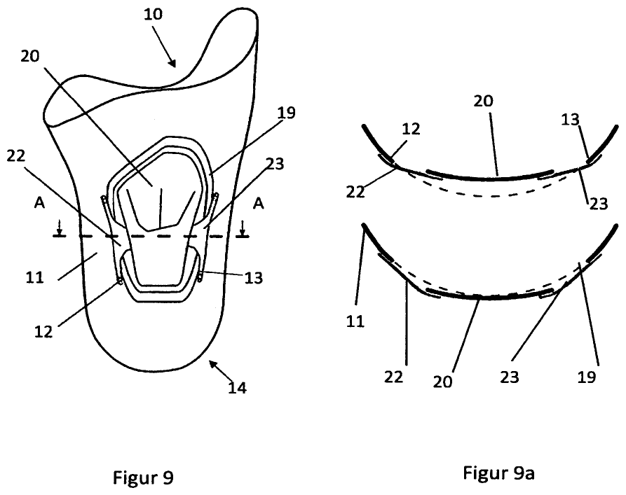

FIG. 8 shows the prosthesis system and its assembly;

FIG. 9 shows a variant with a functional element as a pad; and

FIG. 9a shows two sectional representations along A-A of FIG. 9.

In FIG. 1, a functional element 20 for a prosthesis socket system is shown on its own in a front view, which functional element 20 comprises a base body 25 having a proximal end 21 and a distal end region 24. The base body 25 accommodates mechanical, electrical and also electronic components, for example one or more energy storage devices, one or more sensors, a control unit, at least one communication interface and/or at least one actuator. The mode of operation and the components are explained in more detail below. In the illustrative embodiment shown, the mechatronic functional element 20 has two anchors 22, 23, which are movably and displaceably fastened to the base body 25. The anchors 23 are adjusted relative to each other and relative to the base body 25 by an actuator (not shown), and it is possible for them to be adjusted either on the basis of sensor data or on account of an actuation of buttons or switches in a control panel 26, which is arranged or formed in the proximal, frontal region of the base body 25. The adjustment takes place either alone or in combination. The respective anchors 22, 23 can thus be displaced separately or preferably in the opposite direction to the opposite anchor. In addition to a rectilinear displacement in opposite directions, the anchors 22, 23 can also execute different displacement paths at their distal and proximal end regions, such that, for example, an outward displacement away from the base body 25 is greater in the proximal region than in the distal region. In the distal end region 24, fastening devices 34 in the form of form-fitting elements or force-fitting elements, for example projections, rear projections, clips, webs, snap-on devices or clamping elements are arranged or formed in order to fasten the base body to a further element of the prosthesis socket system. The mechatronic functional element 20 with the anchors 22, 23 can be industrially prefabricated and comprises all the sensors and electrical, electronic and mechanical components necessary for the intended function. Thus, it is possible to provide the mechatronic functional element 20 as a prefabricated module which can be arranged on a respective patient or an individual prosthesis socket and fastened thereto, in order to permit, via the mechatronic functional element, an adaptation, individualization and also functional expansion of a prosthesis socket.

A main body 10 of a prosthesis socket with a socket wall 11 is shown in FIG. 2. The main body 10 has a proximal access opening and a substantially closed distal end region, which is formed as an end cap 14 in the illustrated exemplary embodiment. Fastening devices for further prosthesis components can be arranged on the end cap14, for example receiving adapters for a prosthesis joint or for other prosthetic or orthopedic components such as rails, drives, prosthetic hands, prosthetic feet or the like. Within the uniformly formed socket wall 11, a cutout is formed so as to give two socket wall edges 12, 13, which lie opposite each other. The cutout can be created during the production of the main body 10 or can be separated out after production of an initially circumferentially closed main body 10. Separation can be done by sawing, grinding, drilling, water-jet cutting or other separating methods. The cutout extends from the proximal edge of the main body 10 in the distal direction to the upper edge of the distal end cap 14 and then widens circumferentially into slits extending to the right and left. The slits allow the socket edges 12, 13 to be displaced toward each other and away from each other. The functional element 20 shown in FIG. 1 is inserted inside the cutout. The cutout is correspondingly shaped for this purpose, that is to say the socket wall edges 12, 13 run toward each other and are spaced apart from each other such that the anchors 22, 23 can be fastened thereto or therein.

Fastening devices 18 for the mechatronic functional element 20 are arranged or formed in the proximal region of the end cap 14. The fastening devices 18 are, for example, form-fitting elements or magnets which are designed and arranged corresponding to the fastening devices 34 of the mechatronic functional element 20. When the functional element 20 is inserted into the cutout of the main body 10, the fastening devices 18, 34 can come into engagement with each other and permit locking of the functional element 20 on the end cap 14.

The anchors 22, 23 can be fastened purely mechanically to the main body 10, for example clamped, riveted, screwed, or fixed in clamping rails which are arranged on the anchors 22, 23 and/or on the socket wall edges 12, 13. Alternatively or in addition, the anchors 22, 23 can be fastened to the socket wall edges 12, 13 or in the circumferentially adjacent socket wall regions in a cohesively bonded manner, for example by gluing, welding or other cohesive bonding methods.

The main body 10 can be formed, for example, from fiber composite materials on a molded model of a limb stump, provided with receiving devices for further prosthesis components, and subsequently prepared with regard to the cutout in such a way that the socket wall edges 12, 13 are suitable for being connected to the anchors 22, 23. On account of the anchors 22, 23 being designed to be movable relative to the base body 25, subsequent adjustment can be easily carried out, so that a lower degree of manufacturing precision is sufficient to produce a prosthesis socket with an exact fit. For this purpose, the inner side of the mechatronic functional element 20, that is to say the side facing the patient, is designed according to the basic contour of the limb that is to be received, in particular curved such that it is able to bear as completely as possible on a limb or on a limb stump. The main body 10 can also be created in an additive manufacturing process based on shape data of the limb or of the limb. In particular, the shape data can be obtained by non-contact scanning and converted into a corresponding 3D printing program. On the basis of these data, the main body 10 is then advantageously manufactured at the same time with the cutout and, optionally, with corresponding socket wall edges 12, 13. For example, thickening arrangements or strips can be formed on the socket wall edges 12, 13 so as to facilitate or enable form-fit locking with corresponding rail elements on the anchors 22, 23.

An alternative way of producing the main body 10 is to produce it in the context of an injection molding process, i.e. to produce a pre-assembled, standardized prosthesis socket main body, for example in different size steps, in order then to incorporate the cutout, or to injection-mold such a main body 10 having existing cutouts. It is also possible to join the main body 10 from several prefabricated modules, so that the socket wall 11 consists of several components.

FIG. 3 shows a fully joined prosthesis socket system with main body 10 and the mechatronic functional element 20, in which the anchors 22, 23 are fastened to the socket wall edge regions 16, 17 on both sides of the main body. As mentioned above, the fastening can be effected by form-fit engagement or cohesive bonding, for example by laminating, pouring, injecting or similar. The distal endpiece of the base body 25 extends beyond the proximal edge of the end cap 14 in the distal direction. The lateral slots serve to facilitate an elastic deformation of the socket wall 11 and to permit a widening and a volume reduction of the prosthesis socket for adaptation to the respective patient.

FIG. 4 shows the mechatronic functional element with maximum adjustment of the anchors 22, 23, which are displaced outward from the base body 25 to the maximum extent.

FIG. 5 shows a maximum widened position of the anchors 22, 23 in the mounted state. The base body 25 is further connected to the end cap 14, and the anchors 22, 23 laminated, inserted, glued on, welded in or on or otherwise fixed to the socket wall 11 are moved apart to the maximum extent, such that the socket wall regions 16, 17 expand outward, which is facilitated by the circumferential slots proximal to the end cap 14. The prosthesis socket is in such a state when, for example, the user of the prosthesis socket system wants to get into or out of the prosthesis socket. An open position of this kind can also be adopted to relieve stress and increase comfort.

FIG. 6 shows the main body 10 with the significantly increased inner diameter and inner volume, with the socket wall edges 12, 13 moved apart from each other to the maximum extent.

FIG. 7 shows the mechatronic functional element 20 and its components in more detail and also the electronic connection. In addition to providing structural strength for the rest of the prosthesis socket, the base body 25 also serves as a housing for receiving further components, in particular an actuator 27, which is in the form of an electric motor optionally with gears and a gearwheel, in which toothed racks connected to the anchors 22, 23 engage on opposite sides. If the actuator 27 is operated clockwise, the anchors 22, 23 move apart in opposite directions. If the actuator 27 is operated in the opposite direction, i.e. counter-clockwise, the two anchors 22, 23 move toward each other again in the direction of the base body 25. This movement can be performed via the control panel 26 with the keys. It is also possible that such an actuation of the anchors 22, 23 and thus a change of volume takes place on the basis of sensor data or externally transmitted data. For this purpose, the mechatronic functional element 20 accommodates a control device 28, which is coupled to sensors 29. The sensors 29 can detect, for example, the pressure of the prosthesis socket, a blood flow, muscle activities, stump activities, a gait situation or even an emergency situation. The sensor data are transmitted to the control device 28. There, a corresponding software program is stored which is processed in the CPU of the control device 28 and leads to the activation or deactivation of the actuator 27. In addition, a communication interface 30 to the control device 28 is provided, via which data can be directed, for example, to a mobile data processing device, such as a mobile phone, tablet, computer, or also via the Internet to an external data processing device. The user or an orthopedic technician can then identify the respective status and receive or return data via a wireless connection, for example in order to open or close the prosthesis socket, to perform a massage function, to perform a load-dependent reduction or enlargement of the prosthesis socket or the like. The communication interface 30 enables networking of the prosthesis socket system with external devices, further prosthesis components, medical evaluation points, databases and/or manufacturers, in order to make necessary adjustments or to obtain data for motion analysis. When using a prosthesis, for example, the orthopedic technician can use the pressure sensors to determine whether and where the load is exerted on the limb, whether sufficient blood circulation is present, how high the temperature is, and whether or not the volume ought to be changed via muscle activity.

An automatic adaptation of the prosthesis socket can take place, for example, when getting into the prosthesis socket, when removing the prosthesis socket, when sitting down, driving, during the stance phase or the swing phase when walking, for rotational stabilization during heel strike, or to compensate for increases or decreases in volume.

FIG. 8 shows a variant of the prosthesis socket system. The upper right illustration shows the mechatronic functional element 20 in an embodiment in which there are no anchors arranged displaceably on the base body 25, but instead fastening devices 32, 33, which permit a reversible, mechanical locking with the anchors 22, 23. The fastening devices 32, 33 have in particular form-fitting elements and/or force-fitting elements, with which it is possible to enable a mechanical, repeatedly releasable and lockable fastening of the functional element 20 to the main body 10. The main body 10 is shown in the left-hand illustration with form-fit elements 35 formed in the anchors 22, 23 already arranged on the socket wall edges. The rails 22, 23 are shaped corresponding to the fastening devices 32, 33 and allow mechanical locking of the rails 22, 23 to the fastening devices 32, 33. For this purpose, the fastening devices 32, 33 are pushed onto the rails 22, 23, clipped on and thus locked with form-fit and/or force-fit engagement, optionally still secured via screws, fastening devices, locking elements or the like. The rails 22, 23 likewise run in the proximal-distal direction and allow insertion of the functional element 20 from above and a mechanical locking, such that a limb stump received within the prosthesis socket is securely held about the entire circumference. The mechatronic functional element 20 is arranged in particular laterally or frontally on the main body 10, so that easy accessibility is afforded. This is particularly advantageous in the case of prosthesis sockets for prosthetic knee joints for receiving thigh stumps. Such an arrangement may also be useful in the case of lower-leg stumps since, at these locations, the slightly increased volume of the prosthesis socket resulting from the integrated mechanical, electronic and electrical components does not interfere during use.

With the prosthesis socket system, an auto-adaptive prosthesis socket system with functional expansion by comparison with conventional prosthesis socket systems is achieved via a standardized or individual socket base in the form of the main body 10 in conjunction with adjustable anchors 22, 23. Several functional elements 20 can also be arranged on a main body 10, for example opposite each other, in order to allow a greater variation of volume adjustments. In addition, it is possible to achieve targeted manipulations of specific contact surfaces. The sensors 29 are advantageously arranged within the functional element 20, and the control device 28 can also be coupled to other sensors or external data sources in order to move the actuator 27. Several actuators 27 may be present in order to achieve either an individual adjustment of the anchors 22, 23 relative to each other or a displacement in several directions. The anchors 22, 23 can be twisted and, in addition to a lateral movement, it is also possible to effect a movement in the proximal-distal direction and/or outward or inward, in each case seen from the stump.

FIG. 9 shows a perspective, schematic representation. a prosthesis socket with a main body 10 and a socket wall 11. The main body 10 has a conical basic shape, which widens in the proximal direction from a distal end cap 14. The main body 10 has a substantially closed cross section and completely surrounds the limb stump (not shown) in the assembled and fitted state. Fastening devices (not shown) for further prosthesis components are present on the distal end cap 14, for example for joints or functional elements such as prosthetic hands, prosthetic feet or the like. Within the socket wall 11, a cutout 19 is arranged which was either formed during the original shaping of the main body 10 or was subsequently incorporated into a main body 10 originally completely surrounding the stump. The cutout 19 can be cut out, sawed out or created by other cutting methods, for example. Socket edges 12, 13 lying opposite each other are formed at the cutout 19. The cutout 19 is asymmetrical and has a greater extent in the proximal-distal direction than in the circumferential direction. The two socket wall edges 12, 13, which extend substantially in the proximal-distal direction, lie opposite each other, even if they do not extend parallel to each other. The two socket wall edges 12, 13 may be framed, likewise the remaining edges of the cut-out 19 may be machined or framed, for example fastening devices or form-fitting elements for fastening the anchors 22, 23 can be arranged or formed on or in the socket wall 11. The cutout 19 can be partially or completely closed by a film, a cushion, a textile or another flexible and optionally elastic cover. The orientation of the socket wall edges 12, 13 is freely selectable. On account of the greater variability in the circumferential direction, an orientation in the proximal-distal direction appears to be probably the most common choice

On the outside of the socket wall 11, two anchors 22, 23 are fastened at the socket wall edges 12, 13 or socket edge wall regions, which anchors 22, 23 are able to be fastened form-fittingly and reversibly to the main body 10. The anchors 22, 23 are mounted movably mounted in the mechatronic functional element 20, which is formed in the illustrated embodiment as a so-called pad. The mechatronic functional element 20 integrates the components described above (not shown in any detail), such as energy storage devices, sensors, transmitters, receivers, energy and data interfaces, control devices, processors, data memories, actuators and motors or the like.

FIG. 9a shows two states with differently positioned anchors 22, 23 relative to the central body of the mechatronic functional element 20. In the upper figure, the anchors 22, 23 are moved toward each other, so that a shortening of the effective length between the socket edges or edges 12, 13 of the cutout 19 is effected. In the exemplary embodiment shown, the socket edges 12, 13 are not or are only insignificantly displaced toward each other, so that the central body of the mechatronic functional element 20 is displaced into the interior of the main body 10. The line of curvature of an uninterrupted main body 10 is represented by the dashed line. In such a state, an increased pressure is exerted by the central body on the interior of the main body 10, that is to say on the stump received therein.

If the two anchors 22, 23 are moved in the opposite direction, i.e. displaced away from each other, the effective length of the mechatronic functional element 20 increases between the two edges 12, 13 in the region of the cutout 19, so that the central body is moved outward. This is shown in the lower illustration in FIG. 9a. The inner circumference in the region of the cutout 19, which is at least partially covered by the mechatronic functional element 20, corresponds substantially to the continued circumferential line of the socket wall 11 of the main body 10. Should the stump require further relief, the movement of the anchors 22, 23 outward relative to the central body of the mechatronic functional element 20 can be continued. If the drives or actuators for the displacement of the central body relative to the anchors 22 are operated in the same direction, the central body between the anchors 22, 23 or between the edges 12, 13 of the cutout 19 can be moved. The same also applies, of course, if the cutout or the socket edges 12, 13 form a cutout which extends as far as the proximal edge of the main body 10.

1. A prosthesis socket system, comprising:
a main body with a socket wall, wherein the main body, in an applied state surrounds a limb or a limb stump at least partially and has at least two opposite socket wall edges or at least two socket wall regions,
two anchors, wherein each of the two anchors is arranged in one of the two opposite socket wall edges or in one of the two opposite socket wall regions, and
a mechatronic functional element fastened to at least one of the two anchors.

2. The prosthesis socket system as claimed in claim 1, wherein the mechatronic functional element comprises at least one of
at least one energy storage device,
a sensor,
a control unit,
a communication interface, and
an actuator.

3. The prosthesis socket system as claimed in claim 1 wherein the mechatronic functional element is designed to be able to be reversibly fastened releasably to the at least one of the two anchors.

4. The prosthesis socket system as claimed in claim 1 wherein the mechatronic functional element is designed for moving the two anchors toward each other, or the mechatronic functional element is designed for shifting relative to the two anchors or the socket wall.

5. The prosthesis socket system as claimed in claim 1 wherein the two anchors are designed as form-fit elements and/or force-fit elements.

6. The prosthesis socket system as claimed in claim 1 wherein the two anchors are fastened in or on the main body in a form-fitting, force-fitting, and/or cohesively bonded manner.

7. The prosthesis socket system as claimed in claim 1 wherein the main body is individually shaped from prefabricated modules or preformed.

8. The prosthesis socket system as claimed in claim 1 wherein the two opposite socket wall edges are produced by a separation method.

9. The prosthesis socket system as claimed in claim 1 wherein the two opposite socket wall edges are spaced apart from each other and are at least partially aligned along each other, or widen conically, or curve in a longitudinal extent of the main body.

10. The prosthesis socket system as claimed in claim 1 wherein the two opposite socket wall edges are oriented in a proximal-distal direction or are oriented obliquely to the proximal-distal direction.

11. The prosthesis socket system as claimed in claim 1 wherein the main body is injection-molded, formed of thermoplastic material, additively manufactured, or laminated.

12. The prosthesis socket system as claimed in claim 1 wherein the main body has a conical basic shape, a conical basic shape with an open cross section, or has a cutout.

13. The prosthesis socket system as claimed in claim 1 further comprising an end cap integrally formed with or fastened on the main body.

14. The prosthesis socket system as claimed in claim 13, further comprising fastening devices for the mechatronic functional element arranged on the end cap.

15. The prosthesis socket system as claimed in claim 1 wherein the two opposite socket wall edges are designed to be elastically displaceable toward each other, and/or the socket wall is designed to be reversibly deformable.

16. The prosthesis socket system as claimed in claim 1 further comprising fastening devices for the two anchors are arranged or formed on the mechatronic functional element.

17. The prosthesis socket system as claimed in claim 1 wherein the mechatronic functional element is arranged between the two opposite socket wall edges, in a cutout of the socket wall, or outside of the socket wall.

18. A method for producing a prosthesis socket system as claimed in claim 1, comprising:
producing a main body comprising with a socket wall and socket wall edges,
fastening or forming anchors on the socket wall edges or on socket wall edge regions, and
fastening a mechatronic functional element to the anchors.

19. The method as claimed in claim 18, wherein the main body is produced by an additive manufacturing process, injection-molding, forming from thermoplastic material, or laminating or directly forming on a model or a limb stump.

20. The method as claimed in claim 18 wherein the main body is initially formed with a closed cross section from which a segment is separated to form the socket wall edges.

21. The method as claimed in claim 18 wherein the anchors on the socket wall edges or on the socket wall edge regions, and the main body are connected to each other in a form-fitting, force-fitting, and/or cohesively bonded manner.

22. The method as claimed in claim 18 further comprising locking the mechatronic functional element onto the anchors in a form-fitting and/or force-fitting manner.
